(19) United States
(12) Patent Application Publication
(10) Pub. No.: US 2025/0320098 A1
(43) Pub. Date: Oct. 16, 2025

(54) MAST HYDRAULIC SENSING SYSTEM AND METHOD

(71) Applicant: Toyota Material Handling, Inc., Columbus, IN (US)

(72) Inventors: Thomas Louis Stemnock, Columbus, IN (US); Kevin Partridge, Columbus, IN (US); Victor Matthew Penrod, Columbus, IN (US)

(21) Appl. No.: 19/175,110

(22) Filed: Apr. 10, 2025

Related U.S. Application Data
(60) Provisional application No. 63/632,909, filed on Apr. 11, 2024.

(51) Int. Cl. B66F 9/22; B66F 9/075; F15B 19/00; G01N 33/28; G08B 21/18

(52) U.S. Cl. CPC B66F 9/22; B66F 9/0755; B66F 9/0759; F15B 19/00; G01N 33/2858; G08B 21/182

(57) ABSTRACT

A material handling vehicle including a hydraulic cylinder, a display, and a controller is provided. The hydraulic cylinder includes a barrel, a piston, and a sensor at least partially disposed within the barrel. The controller is in communication with the sensor and the display. The controller is configured to determine whether an output value of an operational parameter of the hydraulic cylinder exceeds a threshold value based on signals from the sensor. If the output value exceeds the threshold, a warning message is shown on the display.

### CROSS-REFERENCE TO RELATED APPLICATION

This application claims priority to U.S. Provisional Patent Application No. 63/632,909, filed Apr. 11, 2024, the entire contents of which are incorporated herein by reference.

### BACKGROUND

A conventional material handling vehicle, such as a forklift, has a mast on its body, a carriage with a load-carrying apparatus (e.g., forks), and a hydraulic cylinder to raise and lower the carriage liftable along the mast. In some instances, the conventional material handling vehicle is provided with multiple sensors, each individually dedicated to monitoring various parameters of the mast, carriage, and hydraulic cylinder (e.g., load weight, mast extension, hydraulic oil pressure, hydraulic oil temperature, hydraulic oil cleanliness, etc.). It can be difficult to collect, manage, process, and/or transmit data from the multiple sensors. Accordingly, it would be useful to provide an improved material handling vehicle with simplified, consolidated, and streamlined data collection for one or more sensors associated with the mast, carriage, the hydraulic cylinder, or a combination thereof.

### SUMMARY

A material handling vehicle that includes a hydraulic cylinder, a mast, a display, and a controller is provided. The hydraulic cylinder includes a barrel, a piston, and a sensor at least partially disposed within the barrel. The mast is coupled to a body of the material handling vehicle and slidably engaged with a carriage. The hydraulic cylinder raises and lowers the carriage along the mast. The display is mounted to the body of the material handling vehicle. The controller is in communication with the sensor and the display and is designed to determine whether an output value of an operational parameter of the hydraulic cylinder exceeds a threshold value based on input data from the sensor. If the output value exceeds the threshold value, a warning message is generated and shown on the display.

In some aspects, the material handling vehicle also has a telematics system that is in communication with a processor of the controller. The controller is configured to transmit the operational parameter of the hydraulic cylinder, the input data from the sensor, the warning message, or a combination thereof to a remote computing device. In some forms, the output value of the operational parameter of the hydraulic cylinder is one of a mast height value, mast stroke cycles, a load weight value, an oil temperature value, an oil pressure value, and an oil cleanliness value, or a combination thereof. In some instances, the oil cleanliness value includes a particulate concentrate of oil in the hydraulic cylinder. In some examples, the warning message includes one or more of: overextension of the hydraulic cylinder, max stroke count exceeded, a load is too heavy, the oil temperature value is too high, the oil pressure value is too high, the oil cleanliness value is poor, speed is too fast, poor stability, and/or poor traction. In some embodiments, the controller is configured to log the warning message in a memory of the controller. In some aspects, the processor of the controller is configured to execute programmable instructions to aggregate the output value of the operational parameter of the hydraulic cylinder of a period of time and analyze a lift and tilt activity of the mast over the period of time. In some forms, the processor also generates preventative maintenance alerts based on the lift and tilt activity of the mast over the period of time and transmits the preventative maintenance alerts to the display. In some instances, the preventative maintenance alerts include stopping points for the mast to be calibrated. In some embodiments, the preventative maintenance alerts include one or more maintenance intervals for an operator to check hydraulic oil within the hydraulic cylinder. In some aspects, the controller is configured to control a lift height of a carriage along a mast based on mast height data received from the sensor and a location of the material handling vehicle received from the telematics system. In some examples, the controller controls the carriage to remain below a lift height threshold. In some examples, the controller controls the carriage to remain within a lift height range. In some examples, the controller controls the carriage to move toa predetermined loading lift height. In some examples, the controller controls the carriage to move to a default travel heigh. In some forms, the material handling vehicle further includes a light assembly movably mounted to the body of the material handling vehicle and communicatively coupled to the controller. The controller is configured to adjust the light assembly as the carriage moves along the mast.

A material handling vehicle that includes a hydraulic cylinder, a mast, a display, a controller, and a telematics system is provided. The hydraulic cylinder includes a barrel, a piston, and a sensor at least partially disposed within the barrel. The mast is coupled to a body of the material handling vehicle and slidably engaged with a carriage. The hydraulic cylinder raises and lowers the carriage along the mast. The display is mounted to the body of the material handling vehicle. The controller is in communication with the sensor and the display and is designed to determine whether an output value of an operational parameter of the hydraulic cylinder exceeds a threshold value based on input data from the sensor. If the output value exceeds the threshold value, a warning message is generated and displayed on the display. The telematics system is communicatively coupled to a processor of the controller and is designed to monitor the material handing vehicle dynamics parameters and vehicle location data.

In some aspects, the material handling vehicle dynamics parameters include one or more of an overall speed of the material handling vehicle, an acceleration of the material handling vehicle, wheel rotational speed values, a steering angle value, a direction of travel, and a wheel slip value. In some forms, the controller restricts a lift height of the carriage along the mast based on a location of the material handling vehicle. In some instances, the controller is configured to send a signal to the hydraulic cylinder to move the carriage along the mast to a predetermined height based on the input data from the sensor.

A method for analyzing operational data regarding a hydraulic cylinder of a material handling vehicle is provided. The method includes determining an output value of an operational parameter of the hydraulic cylinder of the material handling vehicle based on input data received from a sensor disposed within the hydraulic cylinder. The method further includes determining whether the output value of the operational parameter of the hydraulic cylinder exceeds a corresponding threshold value using a processor of a controller coupled to the hydraulic cylinder and the sensor. The method also includes generating a warning message if the output value of the operational parameter of the hydraulic cylinder exceeds the corresponding threshold value. In some instances, a warning message is displayed on a display mounted to the material handling vehicle.

In some aspects, the output value of the operational parameter of the hydraulic cylinder is one of a mast height value, mast stroke cycles, a load weight value, an oil temperature value, an oil pressure value, an oil cleanliness value, or a combination thereof. In some forms, the warning message includes one or more of: overextension of the hydraulic cylinder, max stroke count exceeded, a load is too heavy, oil temperature is too high, oil pressure is too high, oil cleanliness is poor, speed is too fast, poor stability, and/or poor traction.

### BRIEF DESCRIPTION OF THE DRAWINGS

FIG. 1 is a front isometric view of a material handling vehicle according to an embodiment.

FIG. 2 is a block diagram of various electronic components associated with the material handling vehicle of FIG. 1;

FIG. 3 illustrates an exemplary display of the material handling vehicle of FIG. 1; and

FIG. 4 is a flow diagram depicting a method for reporting operational data of the material handling vehicle of FIG. 1.

### DETAILED DESCRIPTION

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize that the examples provided herein have many useful alternatives and fall within the scope of embodiments of the invention.

Before any embodiments of the invention are explained in detail, it is to be understood that the invention is not limited in its application to the details of construction and the arrangement of components set forth in the following description or illustrated in the attached drawings. The invention is capable of other embodiments and of being practiced or of being carried out in various ways. Also, it is to be understood that the phraseology and terminology used herein are for the purpose of description and should not be regarded as limiting. For example, the use of “including,” “comprising,” or “having” and variations thereof herein is meant to encompass the items listed thereafter and equivalents thereof, as well as additional items.

As used herein, unless otherwise specified or limited, the terms “mounted,” “connected,” “supported,” and “coupled” and variations thereof are used broadly and encompass both direct and indirect mountings, connections, supports, and couplings. Further, unless otherwise specified or limited, “connected” and “coupled” are not restricted to physical or mechanical connections or couplings.

As used herein, unless otherwise specified or limited, “at least one of A, B, and C,” and similar other phrases, are meant to indicate A, or B, or C, or any combination of A, B, and/or C. As such, this phrase, and similar other phrases can include single or multiple instances of A, B, and/or C, and, in the case that any of A, B, and/or C indicates a category of elements, single or multiple instances of any of the elements of the categories A, B, and/or C.

As explained above, it would be useful to provide an improved material handling vehicle with simplified, consolidated, and streamlined data collection for at least one or more of a mast, a carriage, and a hydraulic cylinder of the material handling vehicle. More particularly, a material handling vehicle that includes a controller that collects input data from mast sensors and transforms the data into output values (e.g., mast height, carriage load, hydraulic oil temperature, drive stability, hydraulic oil cleanliness, etc.). The output data may be analyzed and/or further processed by the user, additional systems of the material handling vehicle, a remote database, remote computing devices, or a combination thereof.

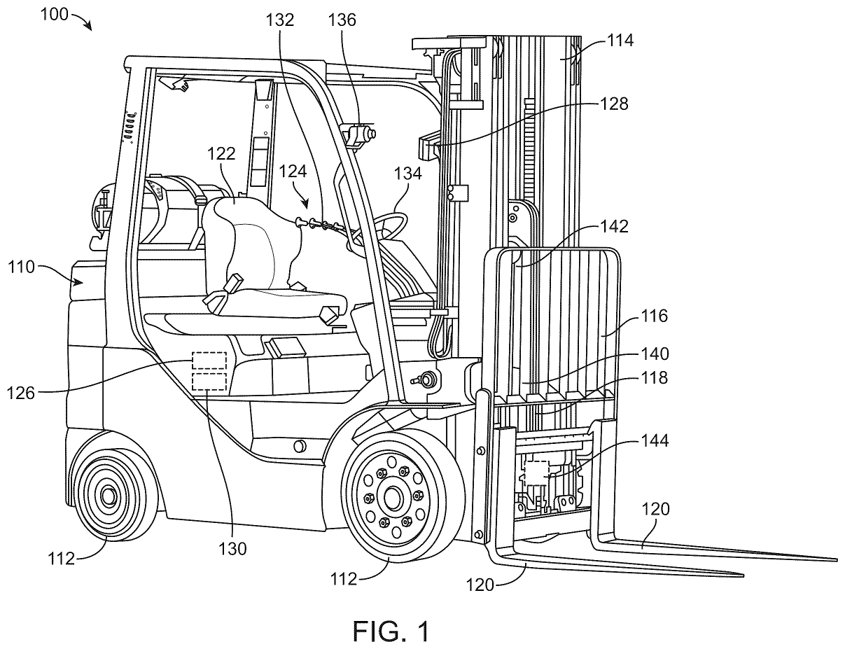

FIG. 1 illustrates a material handling vehicle 100 according to one embodiment. The material handling vehicle 100 includes a body 110, a set of wheels 112, a mast 114, a carriage 116, and at least one hydraulic cylinder 118. The set of wheels 112 is rotatably engaged with the body 110 to move the material handling vehicle 100 about a work area (e.g., warehouse, factory, lumber yard, etc.). The mast 114 is pivotably attached to the body 110 and slidably engaged with the carriage 116. The hydraulic cylinder 118 is supported by the mast 114 and engaged with the carriage 116 to raise and lower the carriage 116 along the mast 114. In some embodiments, the hydraulic cylinder(s) 118 can operate between two sections of the mast 114 to extend the mast 114. In the illustrated example, the carriage 116 is equipped with a set of forks 120 to engage palletized loads. The carriage 116 may be provided with any configuration to engage various loads (e.g., a paper roll clamp, a loaded container handler, etc.). It should also be understood that the hydraulic cylinder 118 may be provided in the form of multiple hydraulic cylinders, including a primary hydraulic cylinder for free lift and one or more secondary hydraulic cylinders for full free lift. In some forms, the hydraulic cylinder 118 also includes one or more tilt cylinders for tilting the mast in the fore and aft directions.

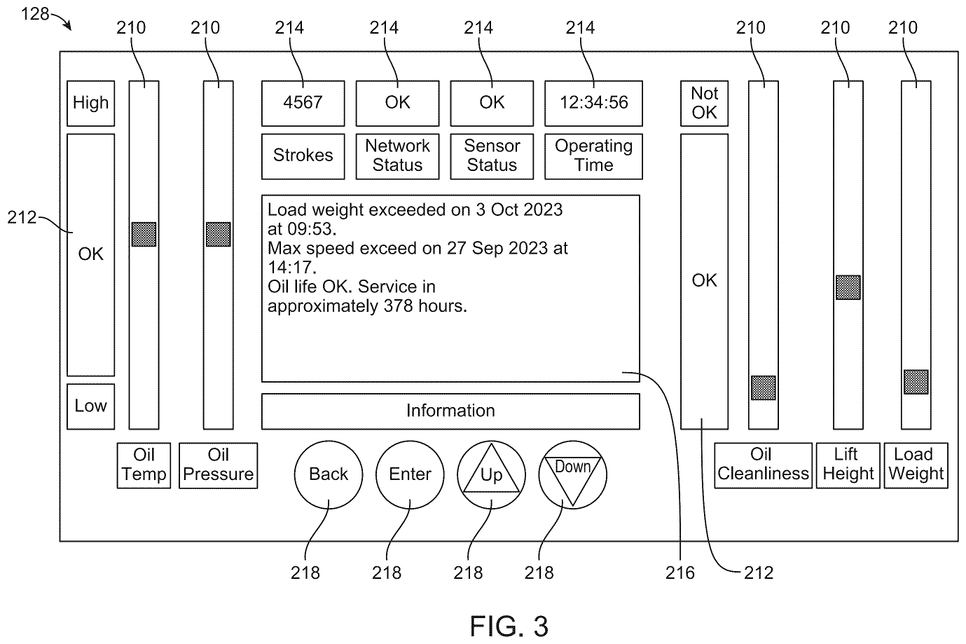

The material handling vehicle 100 further includes an operator seat 122, hand controls 124, a controller 126, a display 128, and a telematics system 130. In the example of FIG. 1, the hand controls 124 include a set of levers 132 and a steering wheel 134. It should be understood that the hand controls 124 may be provided in the form of other or additional control devices such as a joystick, a knob, a handwheel, a button, a switch, a pedal, etc., alternatively or in addition to the illustrated set of levers 132 and steering wheel 134. The material handling vehicle 100 also includes a light assembly 136 moveably mounted to the body 110 and/or the mast 114. The hand controls 124, the display 128, the telematics system 130, and the light assembly 136 are electrically and/or communicatively coupled to the controller 126. The controller 126 processes and communicates operator inputs from the hand controls 124 to the functional equipment of the material handling vehicle 100 (e.g., the set of wheels 112, the mast 114, the carriage 116, the hydraulic cylinder 118, etc.). The controller 126 is also designed to execute one or more commands from the telematics system 130. The display 128 shows information (e.g., operating parameters, equipment statuses, maintenance schedules, preventative maintenance recommendations, location, operator checklists, instructions, warnings, etc.) to the operator. Additional examples of display interface characteristics of the display 128 are described in connection with FIG. 3.

Additionally, the hydraulic cylinder 118 is provided in the form of a barrel 140, a piston 142, and a sensor 144. The barrel 140 slidably receives the piston 142 and supports the sensor 144. More particularly, the sensor 144 is at least partially disposed within the barrel 140. When hydraulic oil fills and is pressurized within the barrel 140, the piston 142 extends out of the barrel 140. When the hydraulic oil is depressurized and drains from the barrel 140, the piston 142 retracts into the barrel 140. Thus, the hydraulic cylinder 118 actuates the carriage 116 upwardly and downwardly along the mast 114 when the barrel 140 is pressurized and depressurized with hydraulic oil. The sensor 144 measures displacement of the piston 142 relative to the barrel 140 and a stroke count associated with the retraction/extension of the piston. Further, the sensor 144 measures oil pressure, oil temperature, and particulate concentration of the hydraulic oil within the barrel 140. As explained in further detail below, the sensor 144 is communicatively coupled to the controller 126. In some forms, the sensor 144 may be provided in the form of a sensor module including multiple sensors or sensing devices.

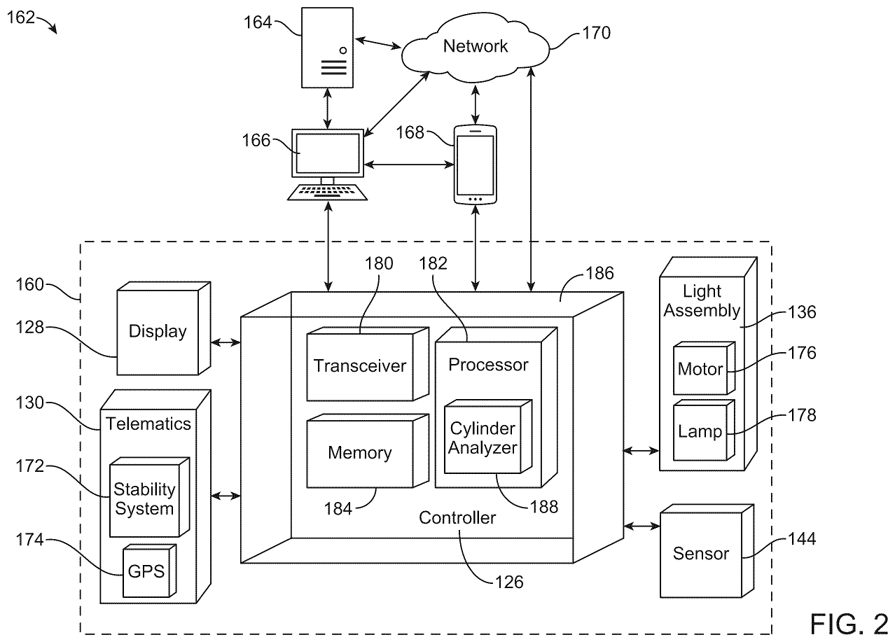

In some instances, using data from the sensor 144 and/or the telematics system 130, the controller 126 restricts lift heights of the carriage 116 and/or moves the carriage 116 to one or more predetermined heights along the mast 114 (e.g., to align with palletized racks, a default travel and/or float height, etc.). In some aspects, the lift height of the carriage 116 may be restricted based on a specific location or zone (e.g., within a warehouse, loading dock, factory, pedestrian zone, charging etc.). In some examples, the controller 126 restricts a lift height of the carriage 116 and/or moves the carriage 116 to predetermined height based on a location detected by one or more sensors of the material handling vehicle 100 (e.g., global positioning system (GPS), RFID, accelerometer, gyroscope, internet of things (IoT), position sensor, etc.), a computer vision system, or a combination thereof. In some instances, using data from the sensor 144 and/or the telematics system 130, the controller 126 restricts a speed, and/or a drive state (e.g., forward, reverse, etc.) of the material handling vehicle 100. In a non-limiting example, the lift height of the carriage 116, a speed of the material handling vehicle 100, and/or a drive state of the material handling vehicle 100 using geofencing. In some instances, predetermined lift heights, lift height ranges, lift height restrictions and/or, lift height thresholds are customizable via the hand controls 124, the display 128, the telematics system 130, the set of levers 132, the steering wheel 134, or a combination thereof. In some instances, the predetermined lift heights, lift height ranges, lift height restrictions and/or, lift height thresholds of the carriage 116 (shown in FIG. 1) are additionally or alternatively customizable via the server 164, the computing device 166, the mobile device 168, and/or the network 170 (see FIG. 2).

Referring in more detail to the sensing and controlling equipment, FIG. 2 illustrates various electronic components 160 associated with the material handling vehicle 100 of FIG. 1 operating in a computing environment 162. The electronic components 160 include the controller 126, the display 128, the telematics system 130, the light assembly 136, and the sensor 144. The computing environment 162 also includes a server 164, a computing device 166, a mobile device 168, and a network 170. The server 164, the computing device 166, and the mobile device 168 are communicatively coupled with one another via the network 170. In some instances, the computing device 166 is directly connected to the server 164 and/or the mobile device 168. The server 164, the computing device 166, the mobile device 168, and the network 170 may be connected to one another wirelessly (e.g., via Wi-Fi, Bluetooth, a cellular network, etc.) and/or via a wired connection (e.g., CAT5, USB, LAN, WAN, etc.).

Further, the telematics system 130 includes a stability system 172 and a GPS unit 174. In some aspects, the GPS unit 174 can be provided in the form of a global navigation satellite system (GNSS) or other location sensing device. The stability system 172 is configured to control certain functional equipment to prevent lateral and longitudinal tipping. For example, the stability system 172 can electronically control the rotational speeds of the set of wheels 112, reduce the tilt speed of hydraulic tilt cylinders, and lock a rear steer axle of the material handling vehicle 100. In some forms, the stability system 172 is part of the controller 126 or comprises a set of functions of the controller 126.

In some forms, the telematics system 130 is provided in the form of one or more telematics devices, including one or more sensors (including the sensor 144 in some aspects), a GPS and/or GNSS unit (including the GPS unit 174 in some aspects), and a communication module to send and receive information with one or more cloud-based platforms (including the server 164, the computing device 166, the mobile device 168, and/or the network 170 in some aspects). The telematics system 130 may be used for fleet management to track a location and status of one or more material handling vehicles in real-time or nearly real-time. The telematics system 130 may be used for operator monitoring including behavior, speed, impacts, safety protocols, checklists, etc. The telematics system 130 may be used to communicate directly with one or more other material handling vehicles and/or other IoT-enabled devices via a gateway, the network 170, or a combination thereof. The telematics system 130 may monitor one or more aspects of a condition of the material handling vehicle 100 and its components. For example, the telematics system 130 can include various sensors designed to receive and process information related to an operation of the material handling vehicle, including but not limited to: temperature, vibration, voltage, speed, acceleration, turn radius, leaks, pressure, etc. to identify when a material handling vehicle 100 is damaged or should undergo preventative maintenance and/or repair. The telematics system 130 may further include access control, impact detection, energy management/monitoring, advanced reporting and analytics, and other data or information related to the operation of the material handling vehicle.

In some aspects, a controller area network (CAN) message may be sent to the telematics system 130 for data processing and collections. The CAN message or messages may contain information requesting customer service for troubleshooting purposes. The CAN message or message may also provide operation threshold information. For example, the message may include a warning if the one or more aspects of the computing environment 162 determines that the material handling vehicle 100 is operating outside of an output value threshold (e.g., mast height value, mast stroke cycles, a load weight value, an oil temperature value, an oil pressure value, an oil cleanliness value, an oil health value, a vehicle speed value, an acceleration value, wheel rotational speed values, a drive stability rating, a steering angle value, a wheel angle value, a direction of travel).

The network 170 may be provided in the form of one or more networks, for example, the Internet, intranets, extranets, wide area networks (WANs), local area networks (LANs), wired networks, wireless networks, cloud networks, or other suitable networks, or any combination of two or more such networks. For example, such networks may include cellular networks, satellite networks, cable networks, Wi-Fi networks, Ethernet networks, RS485 connections, and other types of networks. In some instances, the network 170 may be operating in accordance with a Global System for Mobile Communication (GSM) network, a Code Division Multiple Access (CDMA) network, a Long Term Evolution (LTE) network, an LTE-Advanced (LTE-A) network, an LTE-A Pro network, a New Radio (NR) network, or other systems and radio technologies, including future systems and radio technologies not explicitly mentioned herein. In some instances, the network 170 may be provided in the form of a short-range network (such as Bluetooth, Near-Field Communication (NFC), etc.). In one example, the network 170 may be an isolated private network utilizing a private Internet Protocol (IP) address and limiting access to the network. In some examples, the network 170 may include one or more computing devices that may be arranged, for example, in one or more server banks or computer banks, or other arrangements. In some examples, the server 164 is provided in the form of a cloud-based server.

The network may connect to the server 164, which may be a remote processing module communicatively connected to the material handling vehicle 100 using wireless or wired communication protocols. As used herein, the terms “transmitting,” “receiving,” or “communicating,” when referring to the network 170, may refer to any portion of the network 170 or a network entity (e.g., a base station, a central unit, a distributed unit, a radio unit, etc.) of a radio access network communicating with another device (e.g., directly or via one or more other network entities).

In some aspects, the controller 126 swivels or otherwise adjusts the light assembly 136 relative to the mast 114 as the carriage 116 moves, thus providing steady illumination to the set of forks 120, loads, and/or an environment around the material handling vehicle 100. The light assembly 136 may include a motor 176 that tilts, rotates, or otherwise adjusts a lamp 178. In some aspects, the material handling vehicle 100 may include multiple light assemblies mounted on each side of the body 110, one each side of the mast 114, or multiple light assemblies on other aspects of the material handling vehicle 100. In some forms, the light assembly 136 may adjust automatically in response to one or more signals (e.g., signal from the hydraulic cylinder 118 that the mast is being raised/lowered, object detected from a computer vision system of the material handling vehicle 100, an operator sensor in the operator seat 122 being triggered, a load being added onto the set of forks 120, etc.). In some aspects, the light assembly 136 may include multiple lamps 178. In some examples, the light assembly 136 may be adjusted via the server 164, the computing device 166, the mobile device 168, the telematics system 130, or other aspects of the computing environment 162.

In some instances, the computing device 166 and the mobile device 168 are wirelessly connected to the controller 126, thus permitting a substantially constant data stream from the controller 126 to the computing device 166 and/or the mobile device 168. In some instances, the computing device 166 and the mobile device 168 are connected to the controller 126 via a wired connection through which data from the controller 126 is occasionally downloaded (e.g., while the material handling vehicle 100 refuels and/or recharges). Further, in some instances, the controller 126 is connected to the network 170 wirelessly and/or via a wired connection. The computing environment 162 configuration is designed to facilitate over the air (OTA) updates to the controller 126, the telematics system 130, and other aspects of the material handling vehicle 100. In some instances, the OTA updates can be initiated remotely via the server 164, the computing device 166, the mobile device 168, or another remote user interface.

The computing device 166 and/or mobile device 168 can include any suitable display interface, including but not limited to: desktop computers, laptop computers, servers, tablets, mobile devices, and other web-based interfaces. In some embodiments, the computing device 166 and/or mobile device 168 can be configured to display the one or more dashboards displaying processed or raw data from the telematics system 130 or other aspects of the material handling vehicle 100. The computing device 166 and/or mobile device 168 can be integrated with various third-party applications or other devices and utilize the network 170 and/or servers 164 to provide seamless synchronization among the components of the computing environment 162 and the material handling vehicle 100 (or aspects thereof).

The controller 126 includes a transceiver 180, a processor 182, and a memory 184. In some embodiments, the transceiver 180, the processor 182, and the memory 184 are supported in a housing 186. As discussed above, the controller 126 communicates with the display 128, the telematics system 130, and the sensor 144 via wired communication and/or wireless communication. The transceiver 180 is configured to wirelessly communicate with the computing device 166, the network 170, and the mobile device 168.

The processor 182 is designed to execute programmable instructions, including executing a cylinder analyzer 188. The cylinder analyzer 188 is designed to analyze input data from the sensor 144. The input data includes one or more of hydraulic oil pressure, hydraulic oil temperature data, piston displacement, rotational speed of the wheels 112, tilt speed of the hydraulic cylinders 118, and particulate concentration in the hydraulic oil. Based upon the input data from the sensor 144, the cylinder analyzer 188 generates output values and/or graphics to be shown on the display 128. The output values may also be communicated to the computing device 166, the mobile device 168, and/or the network 170 for display or additional analysis. In some aspects, the input data is compared to one or more threshold values corresponding to an acceptable range for the associated operating parameter, fluid level, pressure, etc. In some forms, the one or more threshold values may be preset by a user and stored in memory 184. In some aspects, the system may automatically determine threshold values based on manufacturer recommendations and/or detected operating parameters of the material handling vehicle 100. In some forms, the controller 126 and/or the telematics system 130 can dynamically adjust the threshold value(s) of one or more aspects of the collected data to improve vehicle performance, improve safety, improve longevity of the vehicle/components(s) or based on other desired outcomes. In some forms, the input data is logged and processed and output values are generated and transmitted for display even if the sensor 144 does not detect one or more values exceeding predefined thresholds.

In one non-limiting example, the output values of the cylinder analyzer 188 can be aggregated and monitored over time to analyze the lift and tilt activity of the mast 114, provide preventative or predictive maintenance alerts to the operator, or provide forensic records to help determine vehicle operation or vehicle damage characteristics before or after an incident. In particular, stopping points for the mast 114 can be calibrated or maintenance intervals for the hydraulic oil can be determined. The output values include but are not limited to one or more of, a mast height value, mast stroke cycles, a load weight value, a hydraulic oil temperature value, a hydraulic oil pressure value, a drive stability rating, an oil cleanliness value, and an overall oil health rating. Additional output values from the processor 182 for communication to the display 128, computing device 166, the mobile device 168, and/or the network 170 include, but are not limited to one or more of, network connectivity status, an operational status of the sensor 144, an elapsed operational time of the material handling vehicle 100, and messages related to the operation of the material handling vehicle 100 (e.g., what loads were lifted, how many loads, distance traveled, route traveled, runtime, charge time, etc.).

Still referring to FIG. 2, the processor 182 may be provided in the form of any suitable processing device or set of processing devices such as, but not limited to a microprocessor, a microcontroller-based platform, a suitable integrated circuit, one or more field programmable gate arrays (FPGAs), and/or one or more application-specific integrated circuits (ASICs). The memory 184 may be provided in the form of any suitable memory type (e.g., volatile memory, non-volatile memory, unalterable memory, read-only memory, high-capacity storage devices, etc.).

The memory 184 is provided in the form of a machine-readable medium on which one or more sets of instructions, such as the software for operating the methods of the present disclosure, can be embedded. The embedded instructions may embody one or more of the methods or logic as described herein. In a particular embodiment, the embedded instructions may reside completely, or at least partially, within any one or more of the memory 184, the computer-readable medium, and/or within the processor 182 during the execution of the instructions.

The terms “non-transitory computer-readable medium” and “tangible computer-readable medium” should be understood to include a single medium or multiple media, such as a centralized or distributed database and/or associated caches and servers that store one or more sets of instructions. The terms “non-transitory computer-readable medium” and “tangible computer-readable medium” also include any tangible medium that is capable of storing, encoding, or carrying a set of instructions for execution by a processor (such as processor 182) or that causes a system to perform any one or more of the methods or operations disclosed herein. As used herein, the term “tangible computer-readable medium” includes any type of computer-readable storage device and/or storage disk and excludes propagating signals.

FIG. 3 illustrates a display interface generated for the display 128 of the material handling vehicle 100. In some embodiments, the display 128 is provided in the form of a touch screen display. In operation, in some embodiments, the illustrative display interface shown includes a graphic with a plurality of indicator bars 210 associated with corresponding qualitative and/or quantitative scales 212. The indicator bars 210 display output values from the cylinder analyzer 188 (e.g., hydraulic oil temperature, hydraulic oil pressure, hydraulic oil cleanliness, mast lift height, load weight, drive stability, overall oil health rating, etc.). The qualitative and/or quantitative scales 212 may have numerical ranges and/or approximate values (e.g., low, within a threshold range, high, outside the threshold range, etc.). The display interface may also show a plurality of indicator windows 214 that display additional output values from the processor 182 (e.g., mast stroke cycles, sensor operational status, network connectivity status, elapsed operational time of the material handling vehicle 100, etc.).

The display interface may also include an information window 216 that provides more detailed information regarding the output values from the cylinder analyzer 188 and the processor 182, preventive maintenance reminders, operational warnings, etc. In some forms, the information window 216 provides information in the form of text-readable narratives, numbers, figures, symbols, or other visually identifiable indicia. The display 128 may further include a plurality of interface buttons 218 (e.g., back, enter, scroll up, scroll down, etc.) to manipulate and navigate the indicator bars 210, the qualitative and/or quantitative scales 212, the indicator windows 214, and/or the information window 216. The interface buttons 218 may be physical buttons, switches, knobs, and/or touch screen buttons. In some embodiments, the indicator bars 210, the qualitative and/or quantitative scales 212, the indicator windows 214, the information window 216, and/or the interface buttons 218 may be rearranged relative to one another on the display 128 or arranged differently than is illustrated in FIG. 3. In some instances, information displayed via the display 128 is transmitted to the server 164, the computing device 166, the mobile device 168, and/or the network 170 via the telematics system 130, or other aspect of the material handling vehicle 100.

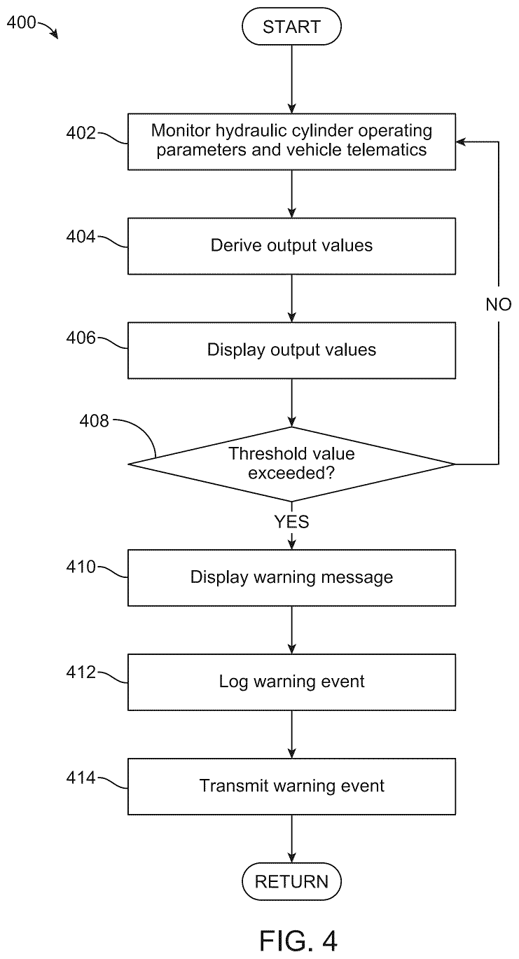

FIG. 4 is a flow diagram depicting a method 400 for displaying and transmitting operational data regarding the material handling vehicle 100 of FIG. 1. The method 400 starts at block 402, where the controller 126 monitors the operating parameters of the hydraulic cylinder 118 based on signals received from the sensor 144 and monitors vehicle telematics parameters based on signals from the stability system 172 and the GPS unit 174 included in the telematics system 130. The operating parameters of the hydraulic cylinder 118 include an extension value of the hydraulic cylinder 118 and a hydraulic pressure value, an oil temperature value, rotational speed of the wheels 112, tilt speed of the hydraulic cylinders 118 and a particulate concentration value of the oil in the hydraulic cylinder 118. In some forms, the extension value of the hydraulic cylinder 118 can include a plurality of extension values, such as a primary cylinder lift extension value, a secondary cylinder lift extension value, or a tilt cylinder extension value. The vehicle telematics parameters include vehicle dynamics parameters and vehicle location data. Vehicle dynamics parameters may include an overall vehicle speed value, an acceleration value, wheel rotational speed values, a steering angle value, a wheel angle value, a direction of travel, a wheel slip value, etc. Vehicle location data may include latitude and longitude, radius and angle from a reference point, position within a predetermined grid, etc. Each of the aforementioned vehicle telematics parameters can be determined based on one or more vehicle sensors that are in communication with the telematics system 130, the controller 126, and/or the processor 182. The method 400 proceeds to block 404.

At block 404, the processor 182 determines output values regarding the operation of the hydraulic cylinder 118 using the cylinder analyzer 188 based on signals received from the sensor 144. Also, the processor 182 determines output values regarding vehicle telematics based on the signals received from the one or more vehicle sensors that are communication with the telematics system 130, the controller 126, and/or the processor 182. For example, the output values can include one or more of a mast height value, mast stroke cycles, a load weight value, an oil temperature value, an oil pressure value, an oil cleanliness value, an oil health value, a vehicle speed value, an acceleration value, wheel rotational speed values, a tilt speed value, a steering angle value, a wheel angle value, a direction of travel, a stability rating, etc. The method 400 proceeds to block 406.

At block 406, the controller 126 displays the output values on the display 128 for reference to an operator of material handling vehicle 100. In some aspects, the data may be logged and/or transmitted regardless of whether corresponding threshold values are met or not. The method 400 proceeds to block 408.

At block 408, the controller 126 determines whether the output values exceed corresponding threshold values that are stored in the memory 184 (e.g., 2.1 meters, 10,000 strokes, 500 kilograms, 50° Celsius, 700 Bar, ISO 4406:1999 20/18/15, 10 km/h, 5 RPM slip, etc.). If, at block 408, the controller 126 determines that none of the output values exceed the corresponding threshold values, the method 400 returns to block 402. However, if, at block 408, the controller 126 determines that one or more output values exceed the corresponding threshold values, the method 400 proceeds to block 410.

At block 410, the controller 126 displays a warning message (e.g., cylinder overextended, max stroke count exceeded, load is too heavy, the oil too hot, the oil pressure too high, the oil is dirty or the oil cleanliness is poor, speed too fast, poor traction, etc.) to the operator via the display 128. In some embodiments, the display 128 may be used to provide general usage data or other telematics data, even when threshold values are not exceeded and/or there are no warnings to display. The method 400 proceeds to block 412.

At block 412, using the cylinder analyzer 188, the controller 126 logs the threshold-exceeding output value, warning message, and corresponding metadata (e.g., location, timestamp, vehicle orientation, vehicle speed, etc.) as a warning event in the memory 184. The method 400 proceeds to block 414.

At block 414, using the transceiver 180, the controller 126 transmits the warning event to the server 164, the computing device 166, the mobile device 168, the network 170, or a combination thereof. The method 400 then returns to block 402.

In other embodiments, other configurations are possible. For example, those of skill in the art will recognize, according to the principles and concepts disclosed herein, that various combinations, sub-combinations, and substitutions of the components discussed above can provide appropriate cooling for a variety of different configurations of motors, pumps, electronic assemblies, and so on, under a variety of operating conditions.

The previous description of the disclosed embodiments is provided to enable any person skilled in the art to make or use the invention. Various modifications to these embodiments will be readily apparent to those skilled in the art, and the generic principles defined herein may be applied to other embodiments without departing from the spirit or scope of the invention. Thus, the invention is not intended to be limited to the embodiments shown herein but is to be accorded the widest scope consistent with the principles and novel features disclosed herein.

What is claimed is:

1. A material handling vehicle, comprising:
a hydraulic cylinder including a barrel, a piston, and a sensor at least partially disposed within the barrel;
a mast coupled to a body of the material handling vehicle and slidably engaged with a carriage, wherein the hydraulic cylinder raises and lowers the carriage along the mast;
a display mounted to the body of the material handling vehicle; and
a controller in communication with the sensor and the display and designed to determine whether an output value of an operational parameter of the hydraulic cylinder exceeds a threshold value based on input data from the sensor, and if the output value exceeds the threshold value, generate a warning message on the display.

2. The material handling vehicle of claim 1 further comprising a telematics system in communication with a processor of the controller, wherein the controller is configured to transmit the operational parameter of the hydraulic cylinder, the input data from the sensor, the warning message, or a combination thereof to a remote computing device.

3. The material handling vehicle of claim 1, wherein the output value of the operational parameter of the hydraulic cylinder is one of a mast height value, mast stroke cycles, a load weight value, an oil temperature value, an oil pressure value, and an oil cleanliness value, or a combination thereof.

4. The material handling vehicle of claim 3, wherein the oil cleanliness value includes a particulate concentrate of oil in the hydraulic cylinder.

5. The material handling vehicle of claim 3, wherein the warning message includes one or more of: overextension of the hydraulic cylinder, max stroke count exceeded, a load is to heavy, the oil temperature value is too high, the oil pressure value is too high, the oil cleanliness value is poor, speed is too fast, poor stability, and/or poor traction.

6. The material handling vehicle of claim 1, wherein the controller is configured to log the warning message in a memory of the controller.

7. The material handling vehicle of claim 1, wherein a processor of the controller is configured to execute programmable instructions to:
aggregate the output value of the operational parameter of the hydraulic cylinder of a period of time; and
analyze a lift and tilt activity of the mast over the period of time.

8. The material handling vehicle of claim 7, wherein the processor is further configured to execute programmable instructions to:
generate preventative maintenance alerts based on the lift and tilt activity of the mast over the period of time; and
transmit the preventative maintenance alerts to the display.

9. The material handling vehicle of claim 8, wherein the preventative maintenance alerts include stopping points for the mast to be calibrated.

10. The material handling vehicle of claim 8, wherein the preventative maintenance alerts include one or more maintenance intervals for an operator to check hydraulic oil within the hydraulic cylinder.

11. The material handling vehicle of claim 2, wherein the controller is configured to control a lift height of a carriage along a mast based on mast height data received from the sensor and a location of the material handling vehicle received from the telematics system.

12. The material handling vehicle of claim 11, wherein the controller controls the carriage to one or more of:
remain below a lift height threshold,
remain within a lift height range,
move to a predetermined loading lift height, and
move to a default travel height.

13. The material handling vehicle of claim 1, further comprising:
a light assembly movably mounted to the body of the material handling vehicle and communicatively coupled to the controller, wherein the controller is configured to adjust the light assembly as the carriage moves along the mast.

14. A material handling vehicle, comprising:
a hydraulic cylinder including a barrel, a piston, and a sensor at least partially disposed within the barrel;
a mast coupled to a body of the material handling vehicle and slidably engaged with a carriage, wherein the hydraulic cylinder raises and lowers the carriage along the mast;
a display mounted to the body of the material handling vehicle;
a controller in communication with the sensor and the display and designed to determine whether an output value of an operational parameter of the hydraulic cylinder exceeds a threshold value based on input data from the sensor, and if the output value exceeds the threshold value, generate a warning message on the display; and
a telematics system communicatively coupled to a processor of the controller and designed to monitor vehicle dynamics parameters and vehicle location data.

15. The material handling vehicle of claim 14, wherein the vehicle dynamics parameters include one or more of an overall speed of the material handling vehicle, an acceleration of the material handling vehicle, wheel rotational speed values, a steering angle value, a direct of travel, and a wheel slip value.

16. The material handling vehicle of claim 14, wherein the controller restricts a lift height of the carriage along the mast based on a location of the material handling vehicle.

17. The material handling vehicle of claim 14, wherein the controller is configured to send a signal to the hydraulic cylinder to move the carriage along the mast to a predetermined height based on the input data from the sensor.

18. A method for analyzing operational data regarding a hydraulic cylinder of a material handling vehicle, comprising:
determining an output value of an operational parameter of the hydraulic cylinder of the material handling vehicle based on input data received from a sensor disposed within the hydraulic cylinder;
determining whether the output value of the operational parameter of the hydraulic cylinder exceeds a corresponding threshold value using a processor of a controller coupled to the hydraulic cylinder and the sensor;
generating a warning message if the output value of the operational parameter of the hydraulic cylinder exceeds the corresponding threshold value; and
displaying the warning message on a display mounted to the material handling vehicle.

19. The method of claim 18, wherein the output value of the operational parameter of the hydraulic cylinder is one of a mast height value, mast stroke cycles, a load weight value, an oil temperature value, an oil pressure value, and an oil cleanliness value, or a combination thereof.

20. The method of claim 18, wherein the warning message includes one or more of: overextension of the hydraulic cylinder, max stroke count exceeded, a load is to heavy, an oil temperature value is too high, oil pressure is too high, an oil cleanliness value is poor, speed is too fast, poor stability, and/or poor traction.
